(19) United States
(12) Patent Application Publication
(10) Pub. No.: US 2026/0294262 A1
(43) Pub. Date: Oct. 1, 2026

(54) VITAL SIGN MONITORING DEVICE

(71) Applicant: ams-OSRAM AG, Premstätten (AT)

(72) Inventor: Daniel RICHTER, Bad Abbach (DE)

(21) Appl. No.: 18/998,169

(22) PCT Filed: Jul. 4, 2023
(86) PCT No.: PCT/EP2023/068337

§ 371 Date: Jan. 24, 2025

(30) Foreign Application Priority Data
Jul. 25, 2022 (DE) 10 2022 118 539.7

(51) Int. Cl. A61B 5/024

(52) U.S. Cl. CPC A61B 5/02427; A61B 2562/0233

(57) ABSTRACT

A vital sign monitoring device includes a single-photon source, a light out-coupling element for coupling light out of the device, a light in-coupling element for coupling light in the device, a controllable delay element to delay light within the device, and a controllable beam splitter. The controllable beam splitter is configured to couple the single-photon source either to the light out-coupling element or to the controllable delay element. The vital sign monitoring device further includes a first photon detector, a second photon detector, and a Hong-Ou-Mandel interferometer. The Hong-Ou-Mandel interferometer has a first input coupled to the controllable delay element, a second input coupled to the light in-coupling element, a first output coupled to the first photon detector, and a second output coupled to the second photon detector.

### TECHNICAL FIELD

The disclosure relates to a vital sign monitoring device using photoplethysmography to examine vital signs, for example the heartrate of a patient.

### BACKGROUND

In the field of vital sign monitoring, a favorable and established measuring method is the use of photoplethysmography (PPG) which is an uncomplicated and inexpensive optical measurement method that allows, for example, heartrate monitoring. PPG is a non-invasive technology that uses a light source in a photon detector at the surface of skin to measure the volumetric variations of blood circulation. For example, the arm/skin is irradiated from light of an incoherent light source, for example an LED, and a simple reflection measurement is performed by means of a photodiode. The influence of the pulsating blood flow on the reflection/absorption behaviour of the photons emitted by the light source is used to draw conclusions about vital parameters, such as heartrate, etc.

The main problem is that the signal-to-noise ratio (SNR) is insufficient for complex measurements, especially when the evaluation of PPT (Pulse Propagation Time) waveform of a photoplethysmogram is necessary. The main reason is that no “depth-resolved” measurement can be made. In this way, refractions from neighboring areas are superimposed on the signal modulated by, for example, blood flow.

There is a desire to provide a vital sign monitoring device which allows “depth-resolved” measurement of vital signs in differently deep layers of the skin.

### SUMMARY

A vital sign monitoring device which provides “depth-resolved” information about vital parameters with low signal-to-noise ratio is specified in claim 1.

A vital sign monitoring device comprises a single-photon source, a light out-coupling element for coupling light out of the device, a light in-coupling element for coupling light in the device, and a controllable delay element to delay light within the device. Furthermore, the vital sign monitoring device comprises a controllable beam splitter being configured to couple the single-photon source either to the light out-coupling element or to the controllable delay element. The vital sign monitoring device further comprises a first photon detector, a second photon detector, and a Hong-Ou-Mandel interferometer. The Hong-Ou-Mandel interferometer has a first input being coupled to the controllable delay element, a second input being coupled to the light in-coupling element, a first output being coupled to the first photon detector, and a second output being coupled to the second photon detector. The controllable delay element is configured to be adjusted for measuring a heartrate with the vital sign monitoring device.

The device allows monitoring of vital signs, such as heartrate or blood volume changes in the microvascular bed of tissue. For this purpose, light is generated by the single-photon source and split into a first and second light portion. The first light portion is fed from the controllable beam splitter to the controllable delay element. Via the controllable delay element, the first light portion reaches the first input of the interferometer. The second light portion is supplied to the light out-coupling element via the controllable beam splitter and penetrates to the outside of the device or into the tissue of a body. The skin is thus irradiated with the second light portion which is partially absorbed and reflected by the various body components to be examined, such as blood or veins.

The reflected part of the second light portion is coupled in the vital sign monitoring device by the light in-coupling element and is fed to the second input of the interferometer. The first light portion, which reaches the interferometer via the controllable delay element at the first input of the interferometer, and the reflected part of the second light portion, which reaches the interferometer at its second input, interpose in the interferometer, and can be detected at the first or second output of the interferometer by means of the first or second photon detector.

By means of the controllable delay element, the signal propagation time of the first light portion within the vital sign monitoring device between the controllable beam splitter and the first input of the Hong-Ou-Mandel interferometer is adjusted to the signal propagation time of the second light portion penetrating the body tissue and thus propagating outside of the vital sign monitoring device between the controllable beam splitter and the second input of the Hong-Ou-Mandel interferometer. By adjusting the delay time of the controllable delay element accordingly, various vital parameters can be measured at different depths of the skin. The vital sign monitoring device therefore enables a “depth-resolved” measurement of vital signs/parameters.

According to a possible embodiment of the vital sign monitoring device, the controllable delay element is configured to be adjusted for measuring the heartrate of a patient as vital sign parameter. For this purpose, in particular, the controllable delay element is configured for adjusting the delay time of the controllable delay element for measuring the heartrate of the patient. This means that the controllable delay element is configured to adjust the delay time of the controllable delay element so that the first light portion which reaches the interferometer at the first input of the interferometer and the reflected part of the second light portion which reaches the second input of the interferometer and which is reflected from the blood veins/vessels or the microvascular bed of tissue superimpose in the interferometer. In conclusion, the vital sign monitoring device allows monitoring the heartrate of a patient by the particular adjustment of the delay time of the controllable delay element.

According to a possible embodiment of the vital sign monitoring device, the single-photon source is configured to emit a pair of a first and second indistinguishable photon. The first photon thus forms the first light portion, and the second photon forms the second light portion.

According to an embodiment of the vital sign monitoring device, the controllable delay element is configured to delay a transfer of the first photon between the controllable beam splitter and the first input of the Hong-Ou-Mandel interferometer within the device.

The first photon detector is configured to detect the occurrence of the first photon and/or the second photon at the first output of the Hong-Ou-Mandel interferometer. The second photon detector is configured to detect the occurrence of the first photon and/or the second photon at the second output of the Hong-Ou-Mandel interferometer.

According to an embodiment, the vital sign monitoring device comprises a control circuit to control the controllable beam splitter and the controllable delay element. The control circuit may be configured to control the controllable beam splitter such that the first photon emitted by the single-photon source is transferred to the controllable delay element, and the second photon emitted by the single-photon source is transferred to the light out-coupling element.

According to an embodiment of the vital sign monitoring device, the control circuit is configured to control the controllable delay element by setting a delay time of the controllable delay element. In particular, the control circuit may set the delay time of the controllable delay element such that the signal propagation time of the first photon in a light guide between the controllable beam splitter and the first input of the Hong-Ou-Mandel interferometer within the device is adapted to the signal propagation time of the second photon penetrating the body tissue outside of the device.

According to an embodiment of the vital sign monitoring device, the control circuit is configured to set the delay time of the controllable delay element such that the first photon is received at the first input of the Hong-Ou-Mandel interferometer and the second photon is received at the second input of the Hong-Ou-Mandel interferometer at the same time, i.e. the signal propagation time of the first photon fed inside of the device between the controllable beam splitter and the first input of the Hong-Ou-Mandel interferometer is adjusted to the signal propagation time of the second photon penetrating the body tissue outside of the device between the controllable beam splitter and the second input of the Hong-Ou-Mandel interferometer.

According to a possible implementation of the vital sign monitoring device, the controllable delay element comprises at least a first and a second delay line for respectively transferring the first photon between the controllable beam splitter and the first input of the Hong-Ou-Mandel interferometer. The first delay line is configured to transfer the first photon between the controllable beam splitter and the first input of the Hong-Ou-Mandel interferometer with another delay time than the second delay line.

According to a possible embodiment, the vital sign monitoring device comprises at least a second controllable beam splitter. The second controllable beam splitter is configured to transfer the first photon between the controllable beam splitter and the first input of the Hong-Ou-Mandel interferometer either via the at least first delay line or via the at least second delay line.

The Hong-Ou-Mandel interferometer is configured to convert the first and second indistinguishable photons into two entangled photons at either the first or the second output of the Hong-Ou-Mandel interferometer, when the first photon arrives at the first input of the Hong-Ou-Mandel interferometer simultaneously with the second photon at the second input of the Hong-Ou-Mandel interferometer.

The single-photon source generates the first and second photon as an indistinguishable photon pair. The first photon is retained from the first input of the interferometer by the controllable delay element as a reference until the second photon, sent towards a measurement point, reaches the second input of the interferometer. The pair of the first and second photon is detected either by the first photon detector at the first output of the Hong-Ou-Mandel interferometer, or by the second photon detector at the second output. Due to quantum mechanical effects, the first and the second photon always exit at the same first or second output of the interferometer if both photons enter the beam splitter of the interferometer through different inputs.

The measurement process is based on quantum mechanical effects. In particular, the special behaviour of a Hong-Ou-Mandel interferometer makes it possible to recognize or separate the relevant information sections under the skin, and to evaluate them specifically. For this purpose, a pair of indistinguishable photons serves as a “measuring pair”. One photon is retained as a reference, and a second photon is sent in the direction of the measuring area.

According to a possible embodiment, the vital sign monitoring device comprises at least a second single-photon source. The single-photon source is configured to generate the pair of the first and second indistinguishable photon with a first wavelength, and the at least one second single-photon source is configured to generate another pair of the first and second indistinguishable photon with at least a second wavelength being different from the first wavelength. Light of different wavelengths selectively interacts with different body components depending on the wavelength. For example, light of a first wavelength is particularly sensitive to haemoglobin, while light of a second wavelength is selective for other blood components, such as iron. The vital sign monitoring device thus enables selective measurement resolution in addition to “depth-resolved” spatial measurement of vital parameters.

According to a possible embodiment, the vital sign monitoring device comprises an evaluation circuit to evaluate the output of the first and second photon detector. In particular, the evaluation circuit may be configured to determine a multiplication of an output signal of the first photon detector and an output signal of the second photon detector, and/or a sum of the output signal of the first photon detector and the output signal of the second photon detector.

According to a possible embodiment of the vital sign monitoring device, the control circuit is configured to control the single-photon source so that a sequence of pairs of the first and second indistinguishable photon is generated by the single-photon source. The control circuit may be configured to set the delay time of the controllable delay element such that, for each pair of the first and second indistinguishable photon, the first photon is received at the first input of the Hong-Ou-Mandel interferometer and the second photon is received at the second input of the Hong-Ou-Mandel interferometer at the same time. The evaluation circuit is configured to evaluate a respective multiplication value for each pair of the first and second indistinguishable photon, each multiplication value representing a result of the multiplication of the output signal of the first photon detector and the output signal of the second photon detector for each pair of the first and second photon. The evaluation circuit is configured to evaluate a histogram of sum values, wherein each sum value represents a result of the sum of the output signal of the first photon detector and the output signal of the second photon detector for each pair of the first and second indistinguishable photon, when it is evaluated by the evaluation circuit that the respective multiplication value for said pair of the first and second indistinguishable photon is below a threshold value.

This embodiment of the vital sign monitoring device enables to perform a first measurement method by creating a histogram of measured sum values. Each measurement value of the histogram is obtained by calculating a sum of the output signal of the first photon detector and the output signal of the second photon detector for each subsequently emitted pair of the first and second indistinguishable photon.

According to another possible embodiment of the vital sign monitoring device, the control circuit is configured to control the single-photon source so that a sequence of pairs of the first and second indistinguishable photon is generated by the single-photon source. The control circuit is configured to set the delay time of the controllable delay element such that, for each pair of the first and second indistinguishable photon, the first photon is received at the first input of the Hong-Ou-Mandel interferometer and the second photon is received at the second input of the Hong-Ou-Mandel interferometer at the same time.

The evaluation circuit is configured to evaluate a respective multiplication value for each pair of the first and second indistinguishable photon. Each multiplication value represents a result of the multiplication of the output signal of the first photon detector and the output signal of the second photon detector for each pair of the first and second indistinguishable photon.

The evaluation circuit is further configured to evaluate an integral of sum values, wherein each sum value represents a result of the sum of the output signal of the first photon detector and the output signal of the second photon detector for each pair of the first and second photon, when it is evaluated by the evaluation circuit that the respective multiplication value for said pair of the first and second photon is low, i.e. below a threshold value.

This embodiment of the vital sign monitoring device enables to perform a second measurement method by continuous integration of the first and second output signals over a plurality of measurement pulses of sequentially emitted photon pairs. A variation in the integral intensity represents a change in the absorption/reflection characteristics in the interaction region.

A third measurement method can be carried out by identifying, in a first step, the relevant depth region under the skin to be examined by setting an appropriate delay time on the delay element and/or by using photons of different wavelengths. In a second step, this depth region is scanned by adjusting the delay with the appropriate wavelength.

Additional features and advantages of the vital sign monitoring device are set forth in the detailed description that follows. It is to be understood that both the foregoing general description and the following detailed description are merely exemplary, and are intended to provide an overview or framework for understanding the nature and character of the claims.

### BRIEF DESCRIPTION OF THE DRAWINGS

The accompanying drawings are included to provide further understanding, and are incorporated in, and constitute a part of, the specification. As such, the disclosure will be more fully understood from the following detailed description, taken in conjunction with the accompanying figures in which:

FIG. 1 shows the basic design of a vital sign monitoring device with signal-to-noise ratio optimization based on two-photon interference, and a possible evaluation of output signals for monitoring vital parameters;

FIG. 2 shows a possible embodiment of the vital sign monitoring device implemented as a photonic on-chip variant based on two-photon interference;

FIG. 3 illustrates a first and second measurement method for monitoring vital parameters by creating a histogram or continuous integration of output signals over several measurement pulses;

FIG. 4 illustrates a third measurement method for monitoring vital parameters by generating photon pairs of different wavelength for monitoring vital signs in different body layers by the vital sign monitoring device; and

FIG. 5 illustrates an embodiment of a vital sign monitoring device comprising a plurality of single-photon sources for generating pairs of first and second indistinguishable photons of different wavelength.

### DETAILED DESCRIPTION OF THE EMBODIMENTS

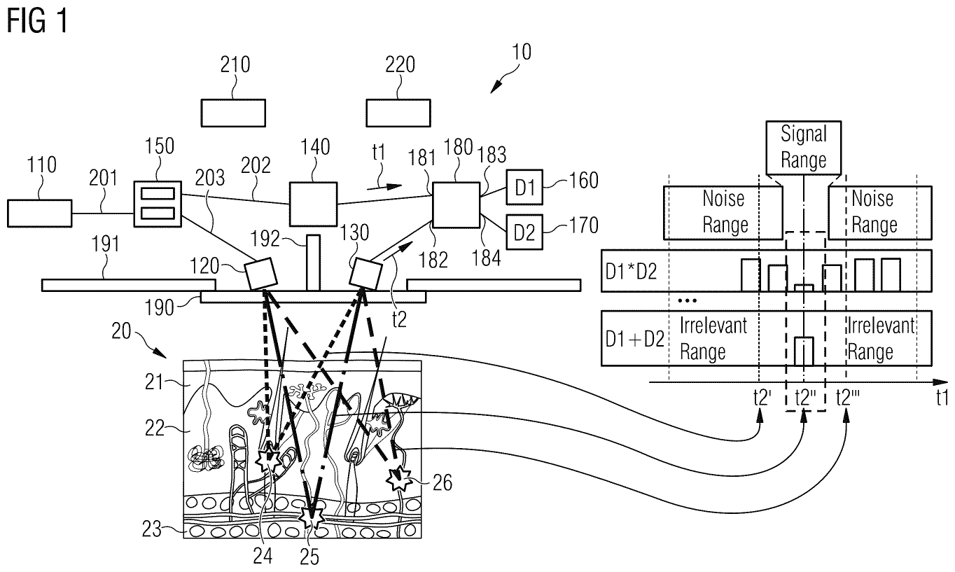

An embodiment of a vital sign monitoring device 10 based on two-photon interference, wherein a pair of indistinguishable photons is generated to record a spatially resolved system response of a body area, for example the skin of a patient, is shown in FIG. 1.

The vital sign monitoring device 10 comprises a single-photon source 110, a light out-coupling element 120 for coupling light out of a window 190 of a housing 191 of the device 10, and a light in-coupling element 130 for coupling light in the device 10 through the window 190. The light in-coupling element 120 and the light out-coupling element 130 are separated by an optical barrier 192. The vital sign monitoring device 10 further comprises a controllable delay element 140 to delay light within the device 10, and a controllable beam splitter 150. The controllable beam splitter 150 is configured to couple the single-photon source 110 either to the light out-coupling element 120 or to the controllable delay element 140.

The vital sign monitoring device 10 further comprises a first photon detector 160, a second photon detector 170, and a Hong-Ou-Mandel interferometer 180. The Hong-Ou-Mandel interferometer 180 has a first input 181 being coupled to the controllable delay element 140, and a second input 182 being coupled to the light in-coupling element 130. The Hong-Ou-Mandel interferometer 180 further comprises a first output 183 being coupled to the first photon detector 160, and a second output 184 being coupled to the second photon detector 170.

The single-photon source 110 is configured to emit a pair of a first and second indistinguishable photon. The second photon may be emitted subsequently to the first photon, or both photons may be generated simultaneously. In particular, the single-photon source 110 is configured to generate a pair of an indistinguishable first and second photon which is transferred by a light guide 201 to the controllable beam splitter 150.

The vital sign monitoring device 10 comprises a control circuit 210 to control the controllable beam splitter 150. The control circuit 210 may be configured to control the controllable beam splitter 150 such that the first photon emitted by the single-photon source 110 is transferred to the controllable delay element 140 via a light guide 202, and the second photon of the photon pair is transferred via a light guide 203 to the light out-coupling element 120.

The controllable delay element 140 is arranged in a light guide 202 between the controllable beam splitter 150 and the Hong-Ou-Mandel interferometer 180. The controllable delay element 140 is configured to delay a transfer of the first photon between the controllable beam splitter 150 and the first input 181 of the Hong-Ou-Mandel interferometer 180.

The control circuit 210 is configured to control the controllable delay element 140 by setting a delay time of the controllable delay element 140. The control circuit 210 is particularly configured to set the delay time of the controllable delay element 140 such that the first photon is received at the first input 181 of the Hong-Ou-Mandel interferometer 180 at the same time as the second photon is received at the second input 182 of the Hong-Ou-Mandel interferometer 180.

The Hong-Ou-Mandel interferometer 180 is configured to convert the first photon and the second indistinguishable photon into two entangled photons at either the first output 183 or the second output 184 of the Hong-Ou-Mandel interferometer, when the first photon arrives at the first input 181 of the Hong-Ou-Mandel interferometer 180 simultaneously with the second photon at the second input 182 of the Hong-Ou-Mandel interferometer 180.

The first photon detector 160 coupled to the first output 183 of the Hong-Ou-Mandel interferometer 180 is configured to detect the occurrence of the first photon and/or the second photon at the first output 183 of the Hong-Ou-Mandel interferometer 180. The second photon detector 170 coupled to the second output of the Hong-Ou-Mandel interferometer 180 is configured to detect the occurrence of the first photon and/or the second photon at the second output 184 of the Hong-Ou-Mandel interferometer 180.

In the following, the operation of the vital sign monitoring device 10 is described.

The single-photon source generates subsequent pairs of the first and second photon such that the first and second photon form an indistinguishable pair that is transferred via light guide 201 to controllable beam splitter 150. Controllable beam splitter 150 is controlled by control circuit 210 so that the first and second photon of each generated pair are split by controllable beam splitter 150 into two different light signal paths 202 and 203.

The second photon of each pair is coupled out of the device 10 through window 190 by light out-coupling element 120 and goes towards skin 20 of a patient to be examined. FIG. 1 shows different layers, such as epidermis 21, dermis 22 and hypoderm 23 of skin 20. Various light rays of the second photons are absorbed or reflected at various components in the different layers of the skin 20. A first light ray of second photons is reflected, for example at the root of a hair 24, a second light ray of the second photons is reflected at blood vessels 25 and a third light ray of the second photons is reflected at a nerve tract 26 within the dermis 22.

The reflected light rays of the second photons enter the device 10 through window 190, and are received by light in-coupling element 130. The reflected second photons are provided from light in-coupling element 130 to the Hong-Ou-Mandel interferometer 180 at a second input 182 of the interferometer. The propagation time of the first photon of each pair of photons is delayed by controllable delay element 140 through controllable beam splitter 150 and Hong-Ou-Mandel interferometer 180 to compensate for the longer propagation time of the second photon of the respective photon pair which interact with the elements in the body tissue to be characterized, for example, blood, veins, haemoglobin, etc.

Both of the first and second photons of each pair are fed to a respective “arm” of Hong-Ou-Mandel interferometer 180, i.e. the first photon of each photon pair is fed to the first input 181 and the second photon of each photon pair is fed to the second input 182 of Hong-Ou-Mandel interferometer 180. By choosing the appropriate delay time of controllable delay element 140, the signal propagation time and thus the investigated depth of skin 20 can be selected.

The vital sign monitoring device 10 comprises an evaluation circuit 220 to evaluate the output of the first and second photon detector 160, 170. In particular, the evaluation circuit 220 is configured to determine a multiplication of an output signal Dl of the first photon detector 160 and an output signal D2 of the second photon detector 170. Furthermore, the evaluation circuit 220 is configured to determine a sum of the output signal D1 of the first photon detector 160 and the output signal D2 of the second photon detector 170.

Since the Hong-Ou-Mandel interferometer 180 converts the first and second photons of each indistinguishable pair of photons into two entangled photons at either the first or the second output 183, 184 of the Hong-Ou-Mandel interferometer 180, each pair of the first and second photon is detected either by the first photon detector 160 at output 183 of the Hong-Ou-Mandel interferometer 180, or by the second photon detector 170 at the second output 184 of the Hong-Ou-Mandel interferometer 180, when the delay time of controllable delay element 140 is properly set so that the first photon of each pair is simultaneously received at the first input 181 of the Hong-Ou-Mandel interferometer 180 as the second photon is received at the second input 182 of the Hong-Ou-Mandel interferometer 180

Referring to FIG. 1, the evaluation circuit 220 detects a minimum of a multiplication value of the output signal D1 of a first photon detector 160 and the output signal D2 of second photon detector 170, if the signal propagation time of the first photon of each pair is properly adjusted by the controllable delay element 140 to the signal propagation time of the second photon of the respective photon pair which is reflected by structures to be investigated in the selected depth of the skin. Thus, the minimum of the multiplication value D1*D2 identifies the desired depth selection of the photons in the skin.

The measurement signal which contains the information of interest of the vital parameters, for example volumetric variations of blood circulation, is coded in a sum value D1+D2 of a sum of the output signal D1 of first photon detector 160 and the output signal D2 of second photon detector 170 for each indistinguishable photon pair. There is also a modulation of the “probe” feedback by for example blood pulsation. To judge on the signal level, a histogram of sum values D1+D2 has to be created for time delays were the multiplication value D1*D2 has a minimum for each pair of the emitted first and second indistinguishable photons. This assures the right photon selection, i.e. only photons from the “right” depth are selected.

Referring to the example shown in FIG. 1, the delay time of the controllable delay element 140 is adjusted such that the first photon of a pair of indistinguishable photons and a second photon of the pair of indistinguishable photons that is reflected at blood vessels 25 of skin 20 arrives at the same time at input 181 and input 182 of Hong-Ou-Mandel interferometer 180. This configuration allows, for example, measuring the heartrate of a patient, because the first photon interferes in the interferometer with the second photon that is reflected at the blood vessels 25.

If signal propagation time t1 of the first photon between controllable beam splitter 150 and first input 181 of Hong-Ou-Mandel interferometer 180 is adjusted to signal propagation time t2 of the second photon, i.e. t1=t2′″, evaluation circuit 220 detects a minimum of the multiplication value D1*D2, i.e. a multiplication value D1*D2 below a threshold level or below multiplication values of adjacent noise areas, in the interested signal range between noise ranges, where t1=t2′ or t1=t2′″.

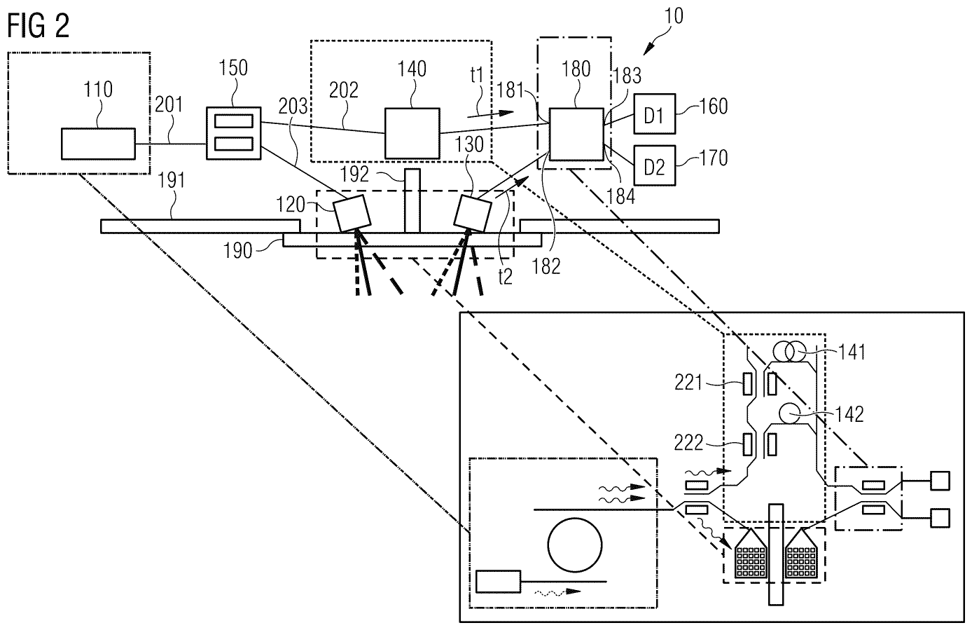

FIG. 2 shows an embodiment of the vital sign monitoring device 10 which is realized as a photonic on-chip variant. The single-photon source 110 may be realized as color centers in the solid, as single atoms or ions in cold traps, as quantum dots or by parametric down-conversion (PDC). All these elements for single photon generation can be realized as photonic integrated circuits.

Referring to FIG. 2, the controllable delay element 140 may comprise at least a first delay line 141 and a second delay line 142 which are respectively configured for transferring the first photon between controllable beam splitter 150 and the first input 181 of the Hong-Ou-Mandel interferometer 180. The first delay line 141 is configured to transfer the first photon between the controllable beam splitter 150 and the first input 181 of the Hong-Ou-Mandel interferometer 180 with another delay time than the second delay line 142. Referring to the example shown in FIG. 2, the second delay line 142 transfers the first photon with a larger delay time than the first delay line 141.

The vital sign monitoring device 10 comprises controllable beam splitters 221, 222. The controllable beam splitters 221, 222 are configured to transfer the first photon between the controllable beam splitter 150 and the first input 181 of the Hong-Ou-Mandel interferometer 180 via the at least first delay line 141 or the at least second delay line 142.

The light out-coupling element 120 and the light in-coupling element 130 may be respectively realized as grating couplers which are separated by an optical barrier. The Hong-Ou-Mandel interferometer 180 may be implemented as a beam splitter.

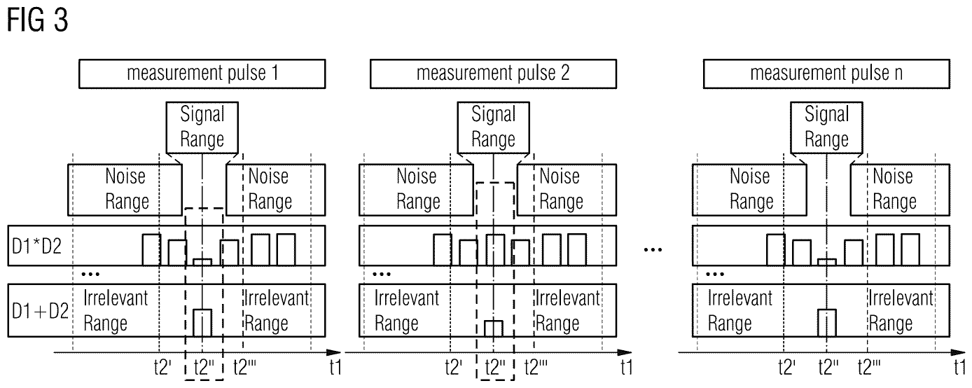

FIG. 3 illustrates possible measurement methods for monitoring vital parameters of the body of a patient to be examined. The output signals D1 and D2 provided by first photon detector 160 and second photon detector 170 are evaluated by evaluation circuit 220. In particular, evaluation circuit 220 may evaluate a multiplication value D1*D2 representing a multiplication of output signal D1 of first photon detector 160 and output signal D2 of second photon detector 170, and/or a sum value D1+D2 representing a sum of output signal D1 of first photon detector 160 and output signal D2 of second photon detector 170.

According to a first measurement method, control circuit 210 controls the single-photon source 110 so that a sequence of pairs of the first and second indistinguishable photon is generated by the single-photon source 110. The delay time of the controllable delay element 140 is set by control circuit 210 such that, for each pair of the first and second indistinguishable photon, the first photon is received at first input 181 of the Hong-Ou-Mandel interferometer 180 at the same time as the second photon is received at the second input 182 of the Hong-Ou-Mandel interferometer 180.

The evaluation circuit evaluates a respective multiplication value for each pair of the first and second indistinguishable photon, wherein each multiplication value represents a result of the multiplication of the output signal of the first photon detector and the output signal of the second photon detector for each pair of the first and second photon. The evaluation circuit 220 evaluates a histogram of sum values, wherein each sum value represents a result of the sum of the output signal D1 of first photon detector 160 and the output signal D2 of second photon detector 170 for each pair of the first and second indistinguishable photon, when it is evaluated by the evaluation circuit 220 that the respective multiplication value for said pair of the first and second indistinguishable photon is below a threshold value.

In the case of a reflected second photon received at second input 182 of Hong-Ou-Mandel interferometer 180, a two-photon interference occurs at the Hong-Ou-Mandel interferometer so that both of the first and second photon of a photon pair are provided at the same output of the interferometer, and the expected minimum of the multiplication value D1*D2 of output signals D1 and D2 is detected by the evaluation circuit 220, as shown for measurement pulse 1 and n, i.e. photon pairs 1 and n, in FIG. 3. On the other hand, if the second photon of a photon pair does not arrive at the second input 182 of Hong-Ou-Mandel interferometer 180, this quantum mechanical effect is not observed, i.e. there is no dip in multiplication value of output signals D1*D2 evaluated by evaluation circuit 220.

An intensity proportional is obtained by an n-fold repetition of the measurement, so that a modulation of the parameter to be measured can be seen in the histogram of sum values of output signals D1+D2, i.e. high and low sum values.

According to a second measurement method illustrated in FIG. 3, the control circuit 210 controls the single-photon source 110 so that a sequence of pairs of the first and second indistinguishable photon is generated by the single-photon source 110. The control circuit 210 sets the delay time of the controllable delay element 140 such that, for each pair of the first and second indistinguishable photon, the first photon is received at the first input 181 simultaneously with the receipt of the second photon at the second input 182 of the Hong-Ou-Mandel interferometer 180.

### The Evaluation Circuit 220 Evaluates a Respective

multiplication value D1*D2 for each pair of the first and second indistinguishable photon. Each multiplication value D1*D2 represents a result of the multiplication of the output signal D1 of first photon detector 160 and the output signal D2 of second photon detector 170 for each pair of the first and second indistinguishable photon. The evaluation circuit 220 then evaluates an integral of sum values D1+D2, wherein each sum value D1+D2 represents a result of the sum of the output signal D1 of first photon detector 160 and the output signal D2 of second photon detector 170 for each pair of the first and second photon, when it is evaluated by the evaluation circuit 220 that the respective multiplication value D1*D2 for said pair of the first and second photon is low, i.e. below a threshold value.

According to this second variant of the measurement method, the evaluation circuit 220 performs a continuous integration of first and second output signals D1 and D2 over a plurality of measurement pulses of sequentially emitted photon pairs. A variation in the integral intensity represents a change in the absorption/reflection properties in the interaction region within a body to be examined.

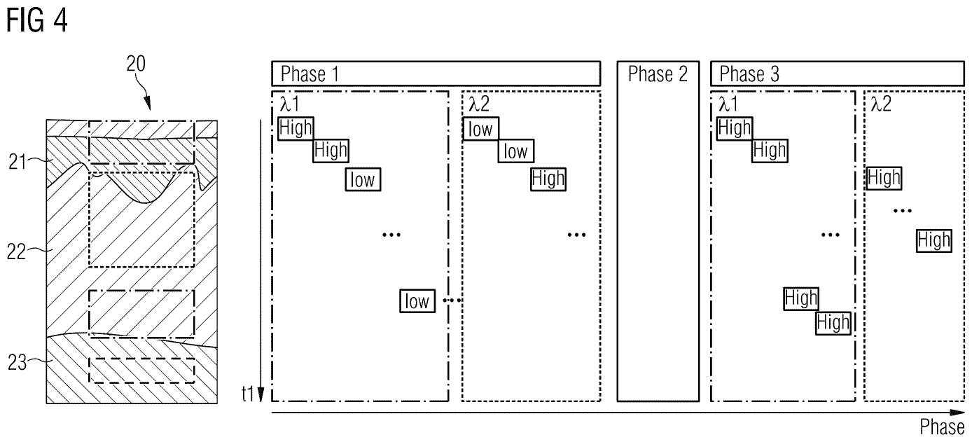

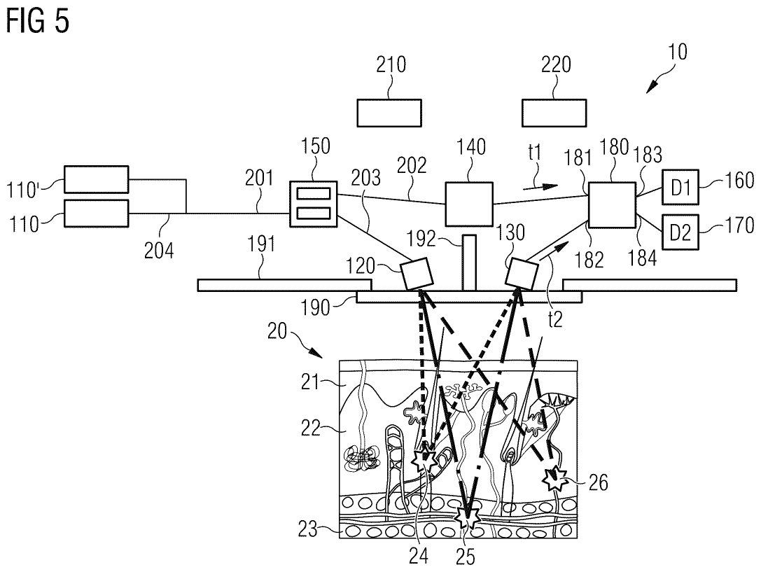

FIG. 4 illustrates another measurement method for monitoring vital parameters within different layers of skin 20 which may be carried out with a vital sign monitoring device 10 as shown in FIG. 5.

The embodiment of the vital sign monitoring device 10 of FIG. 5 comprises the same components as shown and explained for the embodiment of FIG. 1 except that the device of FIG. 5 comprises at least a second single-photon source 110′ in addition to the single-photon source 110. The single-photon source 110 and the at least one second single-photon source 110′ are coupled to the controllable beam splitter by a beam combiner 204. The single-photon source 110 is configured to generate the pair of the first and second indistinguishable photon with a first wavelength. The at least one second single-photon source 110′ is configured to generate another pair of the first and second indistinguishable photon with at least a second wavelength being different from the first wavelength.

According to the embodiment of a measurement method that may be performed with the vital sign monitoring device of FIG. 5, the single-photon sources 110, 110′ are configured to generate the pair of the first and second indistinguishable photon with different wavelengths λ1, λ2, etc. This measurement principle makes use of the fact that different areas/skin layers or structures in the skin show different reflection/absorption behaviour for photons of different wavelengths. FIG. 4 illustrates, for example, that an area in the epidermis 21 and structures in a lower area of the dermis 22 are particularly sensitive for photons with wavelength λ1, whereas an upper area in the dermis 22 is sensitive for photons with wavelength λ2.

In a first phase of the illustrated measurement method, relevant areas in the skin to be investigated can be detected by generating photons of different wavelengths and varying the delay time of controllable delay element 140. In a second phase, the relevant depth in the skin layers and the sensitive wavelengths are derived. The relevant area to be investigated can be scanned by adjusting the signal propagation time of the controllable delay element and generating photons with the appropriate wavelength. This results in a continuously optimized measurement profile in phase 3.

The proposed vital sign monitoring device enables system response from irrelevant areas/noise areas in the skin of a patient to be suppressed. Moreover, the vital sign monitoring device enables a depth/distance resolved provision of vital parameters so that, for example, a differentiation of blood pathways/vessels in different depths of body tissue is possible. In particular, blood response in arteries/veins can be separated from blood response in “normal” vessels. Measured parameters/conditions can be recorded in different body areas, for example oxygen saturation in arteries/veins versus dermis.

Moreover, the vital sign monitoring device 10 allows measurement of vital parameters with improved signal-to-noise ratio, increased precision and accuracy in healthcare parameters, because most of the DC component in a PPG signal is not detected. The PPG waveform comprises a pulsatile (AC) physiological waveform attributed to cardiac synchronous changes in the blood volume with each heartbeat, and is superimposed on a slowly varying (“DC”) baseline with various lower frequency components attributed to respiration, sympathetic nervous system activity and thermal regulation. An improved signal-to-noise ratio also makes a more detailed evaluation of the “PPG waveform” possible, which makes a large number of measurement parameters accessible.

The embodiments of the vital sign monitoring device disclosed herein have been discussed for the purpose of familiarizing the reader with novel aspects of the vital sign monitoring device. Although preferred embodiments have been shown and described, many changes, modifications, equivalents and substitutions of the disclosed concepts may be made by one having skill in the art without unnecessarily departing from the scope of the claims.

In particular, the design of the vital sign monitoring device is not limited to the disclosed embodiments, and gives examples of many alternatives as possible for the features included in the embodiments discussed. However, it is intended that any modifications, equivalents and substitutions of the disclosed concepts be included within the scope of the claims which are appended hereto.

Features recited in separate dependent claims may be advantageously combined. Moreover, reference signs used in the claims are not limited to be construed as limiting the scope of the claims.

Furthermore, as used herein, the term “comprising” does not exclude other elements. In addition, as used herein, the article “a” is intended to include one or more than one component or element, and is not limited to be construed as meaning only one.

This patent application claims the priority of German patent application with application No. 102022118539.7, the disclosure content of which is hereby incorporated by reference.

### REFERENCES

10 vital sign monitoring apparatus
20 skin
21 epidermis
22 dermis
23 hypoderm
24 root of a hair
25 blood vessel
26 nerve tract
110 single-photon source
120 light out-coupling element
130 light in-coupling element
140 controllable delay element
150 controllable beam splitter
160 first photon detector
170 second photon detector
180 Hong-Ou-Mandel interferometer
190 window
191 cover
192 optical barrier
201, 202, 203 light guide/signal path
204 beam combiner
210 control circuit
220 evaluation circuit
D1 output signal of first photon detector
D2 output signal of second photon detector
t1, t2 signal propagation time/delay time

1. A vital sign monitoring device, comprising:
a single-photon source,
a light out-coupling element for coupling light out of the device,
a light in-coupling element for coupling light in the device,
a controllable delay element to delay light within the device,
a controllable beam splitter being configured to couple the single-photon source either to the light out-coupling element or to the controllable delay element,
a first photon detector,
a second photon detector,
a Hong-Ou-Mandel interferometer having a first input being coupled to the controllable delay element, a second input being coupled to the light in-coupling element, a first output being coupled to the first photon detector, and a second output being coupled to the second photon detector,
wherein the controllable delay element is configured to be adjusted for measuring a heartrate with the vital sign monitoring device.

2. The vital sign monitoring device of claim 1, wherein the single-photon source is configured to emit a pair of a first and second indistinguishable photon.

3. The vital sign monitoring device of claim 2, wherein the controllable delay element is configured to delay a transfer of the first photon between the controllable beam splitter and the first input of the Hong-Ou-Mandel interferometer.

4. The vital sign monitoring device of claim 2,
wherein the first photon detector is configured to detect the occurrence of the first photon and/or the second photon at the first output of the Hong-Ou-Mandel interferometer,
wherein the second photon detector is configured to detect the occurrence of the first photon and/or the second photon at the second output of the Hong-Ou-Mandel interferometer.

5. The vital sign monitoring device of claim 2, comprising:
a control circuit to control the controllable beam splitter and the controllable delay element.

6. The vital sign monitoring device of claim 5, wherein the control circuit is configured to control the controllable beam splitter such that the first photon emitted by the single-photon source is transferred to the controllable delay element, and the second photon is transferred to the light out-coupling element.

7. The vital sign monitoring device of claim 5, wherein the control circuit is configured to control the controllable delay element by setting a delay time of the controllable delay element.

8. The vital sign monitoring device of claim 7, wherein the control circuit is configured to set the delay time of the controllable delay element such that the first photon is received at the first input of the Hong-Ou-Mandel interferometer and the second photon is received at the second input of the Hong-Ou-Mandel interferometer at the same time.

9. The vital sign monitoring device of claim 1,
wherein the controllable delay element comprises at least a first and second delay line for respectively transferring the first photon between the controllable beam splitter and the first input of the Hong-Ou-Mandel interferometer,
wherein the first delay line is configured to transfer the first photon between the controllable beam splitter and the first input of the Hong-Ou-Mandel interferometer with another delay time than the second delay line.

10. The vital sign monitoring device of claim 9, comprising: at least a second controllable beam splitter, wherein the second controllable beam splitter, is configured to transfer the first photon between the controllable beam splitter and the first input of the Hong-Ou-Mandel interferometer either via the at least first delay line or the at least second delay line.

11. The vital sign monitoring device of claim 2, wherein the Hong-Ou-Mandel interferometer is configured to convert the first and second indistinguishable photons into two entangled photons at either the first or the second output, of the Hong-Ou-Mandel interferometer, when the first photon arrives at the first input of the Hong-Ou-Mandel interferometer simultaneously with the second photon at the second input of the Hong-Ou-Mandel interferometer.

12. The vital sign monitoring device of claim 2, comprising:
at least a second single-photon source,
wherein the single-photon source is configured to generate the pair of the first and second indistinguishable photon with a first wavelength and the at least one second single-photon source is configured to generate another pair of the first and second indistinguishable photon with at least a second wavelength being different from the first wavelength.

13. The vital sign monitoring device of claim 1, comprising:
an evaluation circuit to evaluate the output of the first and second photon detector,
wherein the evaluation circuit is configured to determine a multiplication of an output signal of the first photon detector and an output signal of the second photon detector and/or a sum of the output signal of the first photon detector and the output signal of the second photon detector.

14. The vital sign monitoring device of claim 13,
wherein the control circuit is configured to control the single-photon source so that a sequence of pairs of the first and second indistinguishable photon is generated by the single-photon source,
wherein the control circuit is configured to set the delay time of the controllable delay element such that, for each pair of the first and second indistinguishable photon, the first photon is received at the first input of the Hong-Ou-Mandel interferometer and the second photon is received at the second input of the Hong-Ou-Mandel interferometer at the same time,
wherein the evaluation circuit is configured to evaluate a respective multiplication value for each pair of the first and second indistinguishable photon, each multiplication value representing a result of the multiplication of the output signal of the first photon detector and the output signal of the second photon detector for each pair of the first and second photon,
wherein the evaluation circuit is configured to evaluate a histogram of sum values, each sum value representing a result of the sum of the output signal of the first photon detector and the output signal of the second photon detector for each pair of the first and second indistinguishable photon, when it is evaluated by the evaluation circuit that the respective multiplication value for said pair of the first and second indistinguishable photon is below a threshold value.

15. The vital sign monitoring device of claim 13,
wherein the control circuit is configured to control the single-photon source so that a sequence of pairs of the first and second indistinguishable photon is generated by the single-photon source,
wherein the control circuit is configured to set the delay time of the controllable delay element such that, for each pair of the first and second indistinguishable photon, the first photon is received at the first input of the Hong-Ou-Mandel interferometer and the second photon is received at the second input of the Hong-Ou-Mandel interferometer at the same time,
wherein the evaluation circuit is configured to evaluate a respective multiplication value for each pair of the first and second indistinguishable photon, each multiplication value representing a result of the multiplication of the output signal of the first photon detector and the output signal of the second photon detector for each pair of the first and second photon,
wherein the evaluation circuit is configured to evaluate an integral of sum values, each sum value representing a result of the sum of the output signal of the first photon detector and the output signal of the second photon detector for each pair of the first and second indistinguishable photon, when it is evaluated by the evaluation circuit that the respective multiplication value for said pair of the first and second indistinguishable photon is below a threshold value.
